(19) United States
(12) Patent Application Publication
(10) Pub. No.: US 2022/0025115 A1
(43) Pub. Date: Jan. 27, 2022

(54) Methods for Measuring Consumption of Amino Acid N-Carboxyanhydrides during Manufacture of Glatiramer Acetate

(71) Applicant: Momenta Pharmaceuticals, Inc., Cambridge, MA (US)

(72) Inventor: Mani S. Iyer, North Chelmsford, MA (US)

(21) Appl. No.: 17/403,278

(22) Filed: Aug. 16, 2021

Related U.S. Application Data
(63) Continuation of application No. 17/136,612, filed on Dec. 29, 2020, now abandoned.
Application No. 17/136,612 is a continuation of application No. 15/135,625, filed on Apr. 22, 2016, now abandoned.
(60) Provisional application No. 62/151,353, filed on Apr. 22, 2015.

(51) Int. Cl. C08G 69/04; C08G 69/10; A61K 47/26; A61K 31/785

(52) U.S. Cl. CPC C08G 69/04; A61K 31/785; A61K 47/26; C08G 69/10

(57) ABSTRACT

Methods for assessing and/or selecting as suitable for commercial release glatiramer acetate, including assessing the consumption, and/or relative reaction kinetics of one or more the amino acid N-Carboxyanhydrides (NCAs) present during the first step of the polymerization reaction in the glatiramer acetate manufacturing process are provided.

### CLAIM OF PRIORITY

This application is a continuation application of, and claims priority to U.S. patent application Ser. No. 17/136,612, titled, “Methods for Measuring Consumption of Amino Acid N-Carboxyanhydrides during Manufacture of Glatiramer Acetate,” filed on Dec. 29, 2020, which is a continuation application of, and claims priority to, U.S. patent application Ser. No. 15/135,625, titled “Methods for Measuring Consumption of Amino Acid N-Carboxyanhydrides during Manufacture of Glatiramer Acetate,” filed on Apr. 22, 2016, which application claims the benefit under 35 U.S.C. § 119(e) of U.S. Provisional Patent Application No. 62/151,353, entitled “Methods for Measuring Consumption of Amino Acid N-Carboxyanhydrides during Manufacture of Glatiramer Acetate,” filed Apr. 22, 2015. The disclosure of the foregoing applications are incorporated herein by reference in their entirety for all purposes.

### TECHNICAL FIELD

The present disclosure relates to methods for assessing, and/or selecting as suitable for commercial release, glatiramer acetate, including assessing the consumption, and/or relative reaction kinetics of one or more the amino acid N-Carboxyanhydrides (NCAs) present during the polymerization reaction in the glatiramer acetate manufacturing process.

### BACKGROUND

Glatiramer acetate (GA), marketed commercially as COPAXONE®, consists of the acetate salts of synthetic polypeptides containing four naturally occurring amino acids: L-glutamic acid, L-alanine, L-tyrosine, and L-lysine with a reported average molar fraction of 0.141, 0.427, 0.095, and 0.338, respectively. Chemically, GA is designated as a polypeptide of L-glutamic acid, L-alanine, L-lysine and L-tyrosine, acetate (salt). Its structural formula is:

(Glu,Ala,Lys,Tyr)ₓ•xCH₃COOH(C₅H₉NO₄•C₃H₇NO₂•C₆H₁₄N₂O₂•C₉H₁₁NO₃)ₓ•xC₂H₄O₂
CAS-147245-92-9

Other than molecular weight and amino acid composition, which are specified in the approved label for the product, the label and other available literature for COPAXONE® does not provide detailed information about the physiochemical characteristics of the product.

### SUMMARY

The present disclosure provides methods for assessing glatiramer acetate or a precursor of glatiramer acetate, and/or for selecting a preparation of glatiramer acetate as suitable formulating as a drug product or as suitable for commercial release and/or selecting a precursor (e.g., Intermediate I) of glatiramer acetate as suitable for use in the manufacture of glatiramer acetate. The methods are based, at least in part, on the levels, consumption, and/or relative reaction kinetics of one or more of the four amino acid N-Carboxyanhydrides (NCAs) present during Step-1, the polymerization reaction, of the glatiramer acetate manufacturing process. As described in more detail below, the methods can include use of high-performance liquid chromatography (HPLC) methods, or equivalent techniques, in combination with amino acid standards for assessing the consumption and relative reaction kinetics of the one more amino acid NCAs in samples of Step-1 material. These assessments can be used, for example and without limitation, to inform whether a batch of Step-1 material (intermediate 1) is suitable for additional processing (for example, proceeding to Step 2 of the manufacturing process) and/or to identify/select a batch of glatiramer acetate as suitable for commercial release (for example, formulating packaging, shipping, releasing into commerce, etc.).

In a specific embodiment, the present disclosure provides methods for manufacturing glatiramer acetate, the method including:
(a) polymerizing in a reaction mixture individual N-carboxy anhydrides (NCAs) of L-alanine (Ala), benzyl-protected L-glutamic acid (Glu), trifluoroacetic acid (TFA)-protected L-lysine (Lys), and L-tyrosine (Tyr) to generate a batch of protected copolymer;
(b) determining the consumption rates of two, three, or all four of the individual NCAs in the reaction mixture; and
(c) processing at least a portion of the batch of the protected copolymer to generate glatiramer acetate if one or more of the following criteria (i)-(v) is determined to have been met in step (b):
   (i) the consumption rate of NCA-Ala is greater than the consumption rate of NCA-Glu, NCA-Lys, and/or NCA-Tyr (Ala>Glu, Lys and/or Tyr);
   (ii) the consumption rate of NCA-Tyr is less than the consumption rate of NCA-Ala, NCA-Glu, and/or NCA-Lys (Tyr<Ala, Glu, and/or Lys);
   (iii) the consumption rates of NCA-Glu and NCA-Lys are less than the consumption rate of NCA-Ala (Ala>(Glu, Lys));
   (iv) the consumption rates of NCA-Glu and NCA-Lys are greater than the consumption rate of NCA-Tyr ((Glu, Lys)>Tyr); and
   (v) the consumption rates of NCA-Glu and NCA-Lys are less than the consumption rate of NCA-Ala and greater than the consumption rate of NCA-Tyr (Ala>(Glu, Lys)>Tyr);

otherwise not processing the batch of protected copolymer to produce glatiramer acetate. In certain embodiments, processing at least a portion of the batch of the protected copolymer to generate glatiramer acetate includes treating the protected copolymer to deprotect the benzyl-protected L-glutamic acid therein and to partially depolymerize the protected copolymer.

In some embodiments of the above method, the step of determining the consumption rate of two, three, or all four of the individual NCAs in the reaction mixture includes obtaining at least one sample of the reaction mixture at two or more time points. The step of determining the consumption rate of two, three, or all four of the individual NCAs in the reaction mixture can include measuring the amount of two, three, or all four of the individual NCAs in at least one obtained sample of the reaction mixture.

In any of the above embodiments, processing the glatiramer acetate to produce glatiramer acetate drug product includes combining the glatiramer acetate with mannitol.

In some embodiments, the dose of the glatiramer acetate drug product is 20 mg. In some embodiments, the dose of the glatiramer acetate drug product is 40 mg. In other embodiments, the dose of the glatiramer acetate drug product is in a range of about 20 mg to about 40 mg. In other embodiments, the dose of glatiramer acetate drug product is about 20 mg, about 25 mg, about 30 mg, about 35 mg, or about 40 mg.

In any of the above embodiments, the method can include filling a syringe with a dose of the glatiramer acetate drug product.

In some embodiments, the step of determining the consumption rate of an individual NCA in at least one collected sample includes determining the amount of the individual NCA that is unreacted. In some embodiments, the step of determining the consumption rate of an individual NCA in at least one collected sample includes derivatizing unreacted NCAs with a detectable label. In some embodiments, the step of determining the consumption rate of an individual NCA in at least one collected sample includes derivatizing NCAs with benzylamine and measuring the amount of benzylamide-derivatized NCA.

In some embodiments of the above methods, the step of determining the consumption rate of an individual NCA in the one or more collected samples comprises the use of at least one standard. In some embodiments, the standard is used to generate a standard curve. In some embodiments of the above methods, the at least one collected sample can be analyzed by Reverse Phase Chromatography.

In some embodiments of the above methods, the detection of benzylamide-derivatized NCA includes ultraviolet (UV) detection.

In some embodiments, the method further includes determining the total consumption of NCAs in the reaction mixture after 24 hours.

In some embodiments of the above methods, in step (a), the NCAs are polymerized using N,N-diethyl amine (DEA) initiator in dioxane solvent.

In some embodiments of the above methods, treating the protected copolymer to deprotect the benzyl-protected L-glutamic acid therein and to partially depolymerize the protected copolymer includes treating the protected copolymer with phenol treated HBr/acetic acid.

In some embodiments, the method includes processing at least a portion of the batch of the protected copolymer to generate glatiramer acetate if two or more of the criteria (i)-(v) set forth above are determined to have been met in step (b), otherwise not processing the batch of protected polymer to produce glatiramer acetate.

In some embodiments, the method includes processing at least a portion of the batch of the protected copolymer to generate glatiramer acetate if three or more of the criteria (i)-(v) set forth above are determined to have been met in step (b), otherwise not processing the batch of protected polymer to produce glatiramer acetate.

In some embodiments, the method includes processing at least a portion of the batch of the protected copolymer to generate glatiramer acetate if four or more of the criteria (i)-(v) set forth above are determined to have been met in step (b), otherwise not processing the batch of protected polymer to produce glatiramer acetate.

In some embodiments, the step of processing the batch of glatiramer acetate includes combining at least a portion of the glatiramer acetate in the batch with a pharmaceutically acceptable carrier or excipient (e.g., mannitol).

In some embodiments: the step of determining the consumption rates of two, three, or all four of the individual NCAs in the reaction mixture comprises determining the consumption rates during the first 2 hours of polymerization reaction; the step of determining the consumption rates of two, three, or all four of the individual NCAs in the reaction mixture comprises determining the consumption rates during the first 5 hours of polymerization reaction; the step of determining the consumption rates of two, three, or all four of the individual NCAs in the reaction mixture comprises determining the consumption rates during a first time period during the polymerization reaction and during a second time period during the polymerization reaction; the samples are obtained during the polymerization reaction and are analyzed after the polymerization reaction is terminated; and at least a portion of the batch of protected copolymer is deprotected and partially depolymerized if one or more of the following criteria (i)-(v) is determined to have been met in step (b).

As used herein, a “protected copolymer” is an intermediate produced by polymerizing in a reaction mixture individual N-carboxy anhydrides (NCAs) of L-alanine (Ala), benzyl-protected L-glutamic acid (Glu), trifluoroacetic acid (TFA)-protected L-lysine (Lys), and L-tyrosine (Tyr).

As used herein, the “consumption rate” of an N-carboxy anhydride (NCA)-modified amino acid during Step 1 of the GA manufacturing process (described below) means rate of consumption at time point during the polymerization reaction following initiation of the polymerization reaction of NCA-derivatized forms of L-alanine, benzyl-protected L-glutamic acid, trifluoroacetic acid (TFA) protected L-lysine and L-tyrosine (collectively referred to as NCAs) to result in a protected copolymer (Intermediate-1). The “amount” of a particular NCA-derivatized amino acid can be expressed as percentage (%) of the total starting material (the starting amount of free NCA-derivatized amino acid) and/or as mole fraction or mole percent fraction of the total amount of free NCA-derivatized amino acid or in any other suitable terms.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram depicting Step 1 of the glatiramer acetate manufacturing process, in which N-carboxy anhydrides (NCA) polymerization is initiated using diethylamine in dioxane to generate Intermediate-1.

FIG. 2 is a schematic diagram depicting the derivitization of an NCA with benzylamine.

FIG. 3 is a graph plotting the percentage of NCA remaining (Y-axis) for each of the indicated amino acids as a function of reaction time (hours, X-axis).

FIG. 4 is a graph plotting NCA depletion as a function of reaction progress with each NCA expressed as a mole fraction of total NCAs.

FIG. 5 is a graph plotting the % NCA Consumption (Y-axis) over time (hours, X-axis) in the Step-1 polymerization reaction run in acetonitrile instead of dioxane.

### DETAILED DESCRIPTION

Manufacture of Glatiramer Acetate

Processes for the manufacture of Glatiramer Acetate (GA) generally include:

Step 1: Polymerization of N-carboxy anhydrides of L-alanine, benzyl-protected L-glutamic acid, trifluoroacetic acid (TFA) protected L-lysine and L-tyrosine (collectively referred to as NCAs) to result in a protected copolymer (Intermediate-1);

Step 2: Depolymerization and benzyl deprotection of Intermediate-1 using, for example, hydrobromic acid in acetic acid (e.g., phenol treated 33% HBr/acetic acid) to generate Intermediate-2; and

Step 3: Deprotection of the TFA-protected lysines on Intermediate-2 (e.g., by treatment with aqueous piperidine) to create Intermediate-3, followed by processing to generate GA and further purification and drying of the isolated GA drug substance.

During the polymerization step, NCAs are co-polymerized in a predetermined ratio using diethylamine as an initiator. In a specific embodiment, the NCAs are polymerized using N,N-diethyl amine (DEA) initiator in dioxane solvent. The addition of diethylamine to the reaction mixture results in modification of the C-terminus of a portion of amino acids in the reaction mixture to DEA. Upon substantially complete consumption of the NCA components, the reaction mixture is quenched in water. The resulting protected polymer (Intermediate-1) is washed with water prior to further processing. The progress of the polymerization step can be monitored by measuring the viscosity of the reaction mixture and the viscosity measurement can be used to determine when the polymerization reaction should be quenched, as described in U.S. Pat. No. 8,575,198.

During depolymerization and benzyl deprotection, Intermediate-1 is treated with phenol-treated 33% HBr in acetic acid (HBr/AcOH). This results in the cleavage of the benzyl protecting group on the glutamic acids as well as cleavage of peptide bonds throughout the polymer. After a period of time the reaction is quenched with water, and the product polymer is isolated by filtration and washed with water. The product polymer, Intermediate-2, has a reduced molecular weight relative to Intermediate-1. Intermediate-2 is washed with water before proceeding to deprotection of TFA-protected lysine. During deprotection TFA-protected lysines, Intermediate-2 is dissolved in aqueous piperidine to remove the trifluoroacetyl group on the lysine. The resulting copolymer, Intermediate-3, is subsequently purified using diafiltration/ultrafiltration and the resulting acetate salt is dried to produce glatiramer acetate drug substance.

Glatiramer acetate drug substance is dissolved in water and combined with mannitol to produce glatiramer acetate drug product.

Exemplary methods for the manufacture of GA are known in the art (see, for example, U.S. Pat. No. 3,849,550; WO 95/031990, US 2006/0154862, US 2007/0021324, US 2010/0256039, US 2007/0021324, US 2009/0263347, U.S. 2010/0256039, and WO 2010/017292).

There are certain detectable attributes of GA, intermediates in the manufacture or GA and aspects of the process for making GA that are conserved from batch-to-batch during the manufacturing process. These attributes can be used, e.g., to select GA or polymeric precursors of GA and/or to monitor, assess, and/or evaluate GA process and/or batch quality. As described herein, the present disclosure provides methods of assessing GA that include determining the relative consumption rates of two, three, or all four of the individual NCAs (N-carboxyanhydrides) of Ala, Glu, Lys and Tyr (in assessing the individual NCAs, it is benzyl-protected Glu-NCA and TFA-protected Lys-NCA that are assessed) present in a reaction mixture following polymerization of those NCA amino acids. As disclosed herein, the present disclosure provides methods of assessing GA that include determining the consumption rates of one, two, three, or all four of the individual NCAs (N-carboxyanhydrides) of Ala, Glu, Lys and Tyr present in a reaction mixture following polymerization of those NCA amino acids. In certain embodiments of the method, at least a portion of the batch of the protected copolymer is processed to generate glatiramer acetate if the consumption rate of Ala is greater than the consumption rate of Glu, Lys, and/or Tyr (Ala>Glu, Lys and/or Tyr) and/or if the consumption rate of Tyr<Ala, Glu, and/or Lys, and/or if the consumption rate of Ala>(Glu, Lys), and/or if the consumption rate of (Glu, Lys)>Tyr, and/or if the consumption rates of each of Glu and Lys are less than the consumption rate of Ala and greater than the consumption rate of Tyr (Ala>(Glu, Lys)>Tyr).

The method may be performed shortly after a sample of the polymerization reaction is obtained or the method can be performed at a later time during the GA manufacturing process. Thus, the samples can be saved (e.g. stored, e.g., frozen) for later analysis, e.g., at any time during subsequent manufacturing and/or processing and/or formulating steps, and/or prior to release (e.g., commercial release) of the GA drug product. In some cases a reference sample is obtained at the time of the initiation of the polymerization reaction.

Thus, for example, also provided herein is a method for identifying a batch of GA as suitable for commercial release. The method can include (a) determining the consumption rates of one, two, three or all four of the individual NCAs selected from the group consisting of L-alanine (Ala), benzyl-protected L-glutamic acid (Glu), trifluoroacetic acid (TFA)-protected L-lysine (Lys), and L-tyrosine (Tyr) in a sample, wherein the sample was obtained from a polymerization reaction mixture of all four of said individual NCAs, the polymerization reaction generating a batch of protected copolymer; (b) identifying a batch of glatiramer acetate produced from the protected copolymer analyzed in step (a) as suitable for commercial release (or formulation in a drug product containing GA and mannitol) if one or more, two or more, three or more, four or more, or all five of the following criteria (i)-(v) is/are determined to have been met in step (b):
(i) the consumption rate of NCA-Ala is greater than the consumption rate of NCA-Glu, NCA-Lys, and/or NCA-Tyr (Ala>Glu, Lys and/or Tyr);
(ii) the consumption rate of Tyr is less than the consumption rate of NCA-Ala, NCA-Glu, and/or NCA-Lys (Tyr<Ala, Glu, and/or Lys);
(iii) the consumption rates of NCA-Glu and NCA-Lys are less than the consumption rate of NCA-Ala (Ala>(Glu, Lys));
(iv) the consumption rates of NCA-Glu and NCA-Lys are greater than the consumption rate of NCA-Tyr ((Glu, Lys)>Tyr); and
(v) the consumption rates of NCA-Glu and NCA-Lys are less than the consumption rate of NCA-Ala and greater than the consumption rate of NCA-Tyr (Ala>(Glu, Lys)>Tyr);

otherwise not identifying the batch of glatiramer acetate produced from the protected copolymer analyzed in step (a) as suitable for commercial release (or not suitable for formulating as a drug product containing GA and mannitol).

Alternatively or in addition, methods disclosed herein can include determining the total consumption of one or more of the NCAs or the relative rate of consumption of two or more NCAs in the reaction mixture at any suitable time, for example, at or after 1, 2, 3, 4, 5, 6, 7, 8, 9, 10, 11, 12, 13, 14, 15, 16, 17, 18, 19, 20, 21, 22, 23, 24 hours or 48 hours.

Determining the consumption rate of one or more NCAs can include determining the rate within the first 2 hrs after initiation of the polymerization reaction, during the first 5 hrs, after initiation of the polymerization reaction or during any other suitable portion of the polymerization reaction. In some cases the samples are obtained during these time periods, but are not analyzed until a later time period.

The methods can also include assessment of the consumption rates of one, two, three, or all four of the individual NCAs of Ala, Glu, Lys, and Tyr, and using, having, storing, and/or providing information resulting from such assessment, e.g., for the selection, review (e.g., quality control), and/or manufacture of GA and/or polymeric precursors of GA. In some embodiments, the present disclosure includes making recommendations for the selection, review (e.g., quality control), and/or manufacture of GA and/or polymeric precursors (e.g., Intermediate 1) of GA based on the methods provided herein.

When one or more the specified criteria are met the method can include: providing and/or receiving information regarding the relative consumption rates or absolute consumption rate to another party (e.g., a party manufacturing GA), classifying, selecting, accepting, discarding, releasing, or withholding a batch of GA; reprocessing a batch through a previous manufacturing step; processing a batch of GA into drug product, shipping the product from a batch of GA, moving the batch of GA to a new location; or formulating, labeling, packaging, selling, offering for sell, releasing a batch of GA into commerce and/or directing any of the above actions.

In some embodiments, the consumption rates of two, three, or all four of the individual NCAs of Ala, Glu, Lys, and Tyr in a batch of protected copolymer can be assessed, and/or compared with the consumption rates of two, three, or all four of the individual NCAs of Ala, Glu, Lys, and Tyr in another sample (e.g., reference sample of Intermediate 1 disclosed herein), and/or with a reference value or standard.

In some embodiments, action may be taken if the consumption rates of one, two, three, or all four of the individual NCAs of Ala, Glu, Lys, and Tyr in a batch of protected copolymer comply with or are consistent with a defined or approved standard. Thus, in some cases it is not necessary to determine relative rates of consumption, but rather depend in determining whether the rate of consumption of a particular NCA conforms to a reference consumption rate

In some embodiments, methods include comparison of the relative consumption rates of two, three, or all four of the individual NCAs of Ala, Glu, Lys, and Tyr in a batch of protected copolymer with defined amounts or values, standards or levels of the consumption rate (e.g., Federal Drug Administration (FDA) approved or commissioned FDA approved values, standards or levels). Data points can be expressed and/or compared as ratios, levels, relative levels, rates, percentages, etc., as long as the data points can be compared. In some cases, conversion of data points can be performed to facilitate comparison.

As described above, the present disclosure provides methods for accurately and/or precisely assessing (e.g., measuring, analyzing, detecting, determining, evaluating, estimating, and/or predicting) the consumption rates of two, three, or all four of the individual NCAs of Ala, Glu, Lys, and Tyr in a batch of protected copolymer. Methods can include comparison of the consumption rates of two, three, or all four of the individual NCAs of Ala, Glu, Lys, and Tyr in a batch of protected copolymer to a reference standard for a protected copolymer precursor (e.g., Intermediate 1) of GA (e.g., a reference standard showing the consumption rates of two, three, or all four of the individual NCAs of Ala, Glu, Lys, and Tyr in a batch of Intermediate-1 of GA). Because the consumption rates of two, three, or all four of the individual NCAs of Ala, Glu, Lys, and Tyr in a batch of protected copolymer is a characteristic, hallmark, and/or signature (e.g., a structural signature) of the GA production process (as demonstrated in the present Examples), information pertaining to the consumption rates of two, three, or all four of the individual NCAs of Ala, Glu, Lys, and Tyr in a batch of protected copolymer can be used to select, monitor, assess, and/or evaluate a GA process and/or batch quality.

In some embodiments, the step of determining the consumption rate of an individual NCA in at least one collected sample, as disclosed herein, includes determining the amount of the individual NCA that is unreacted. In some embodiments, the step of determining the consumption rate of an individual NCA in at least one collected sample includes derivatizing unreacted NCAs with a detectable label. For example, as described in Example 1, in some embodiments, the step of determining the consumption rate of an individual NCA in at least one collected sample includes derivatizing NCAs with benzylamine and measuring the amount of benzylamide-derivatized amino acids. Benzylamide derivatized amino acids can be detected, for example by UV detection, using, e.g., HPLC.

Methods discussed herein can be used to identify differences in GA materials not observed using conventional methods. For example, by evaluating the consumption rates of one, two, three, or all four of the individual NCAs of Ala, Glu, Lys, and Tyr in a batch of Intermediate 1 produced during the GA manufacturing process, one can identify non-conforming compositions during or following the GA manufacturing process.

Alternatively or in addition, methods can be used to confirm consistency between initiation/propagation kinetics in GA production processes (e.g., to determine equivalence or compare consistency in initiation kinetics between lots of material). For example, comparisons can be made between samples of two or more different batches of Intermediate 1 used to produce different batches or lots of GA, e.g., manufactured by different manufacturers, two or more batches or lots of GA manufactured at different locations, two or more batches or lots of GA manufactured at different times, two or more batches or lots of GA manufactured different processes, and/or two or more batches or lots of GA manufactured using altered or modified initiation kinetics. The compositions and methods herein can also be used in quality control, e.g., to compare and/or confirm batch-to-batch consistency between lots made by a consistent process.

In some embodiments, the consumption rates of individual NCAs of two or more of Ala, Glu, Lys and Tyr in a polymerization reaction (e.g., whether determined, provided, or obtained) can be compared to the values provided in Table 1. In some instances, the consumption rates of individual NCAs of two or more of Ala, Glu, Lys and Tyr in a polymerization reaction can correlate with, can be equal (e.g., about equal), and/or can be equivalent (e.g., about equivalent) to the relative consumption rates shown in Table 1:

TABLE 1
Reference Specifications for Relative NCA Consumption Rates

| Reference Specification | Percent consumption of individual NCAs |
|---|---|
| i | Ala > Glu, Lys and/or Tyr |
| ii | Tyr < Ala, Glu, and/or Lys |
| iii | Ala > (Glu, Lys) |
| iv | (Glu, Lys) > Tyr |
| v | Ala > (Glu, Lys) > Tyr |

Table 1 shows the relative consumption rates of two, three or four NCAs following the polymerization reaction in Step 1 of the GA manufacturing process. As disclosed herein, a portion of a batch of protected copolymer may be processed to generate glatiramer acetate if one or more of the criteria in Table 1 is/are determined to have been met. As shown in Table 1, and as used throughout this disclosure, “(Glu, Lys)” means that the consumption rates for each of Glu and Lys are not being compared to each other, but only to one or more other consumption rates of other amino acids. Thus, for example, (Glu, Lys)>Tyr means that the rate of Glu consumption can be higher or lower than, or the same as, the rate of Lys consumption, so long as the consumption rates of each of Glu and Lys is greater than that of Tyr.

In some instances, the relationships shown in Table 1 are reference specifications for commercial release of GA, and the present methods can include comparing the relative consumption rates of two, three or four NCAs following the polymerization reaction in Step 1 of the GA manufacturing process to one or more of the reference specifications (i)-(v) in Table 1. For example, the methods herein generally include polymerizing in a reaction mixture individual NCAs of L-alanine (Ala), benzyl-protected L-glutamic acid (Glu), trifluoroacetic acid (TFA)-protected L-lysine (Lys), and L-tyrosine (Tyr) to generate a batch of protected copolymer; determining the consumption rates of two, three, or all four of the individual NCAs in the reaction mixture; and processing at least a portion of the batch of the protected copolymer to generate glatiramer acetate if one or more of the reference specifications in Table 1 are met.

The methods can be used, for example, to select a sample of GA or a polymeric precursor thereof (e.g., Intermediate-1) for further use; select (e.g., as suitable for sale or for administration (e.g., injection to a human) a sample of GA or a polymeric precursor thereof (e.g., Intermediate-1); classify, accept, release, process into drug product a sample of GA; select a sample of GA or a polymeric precursor thereof (e.g., Intermediate-1) for shipment, moving to a new location, formulating, labeling, packaging, selling, offering for sale, releasing into commerce; and/or selecting a sample of GA or a polymeric precursor thereof (e.g., Intermediate-1) for use in a manufacturing process for GA.

In some cases the consumption rate of each amino acid (NCA-amino acid) is based on the percentage of the original amount of the amino acid in the polymerization reaction at the outset of polymerization (i.e., the consumption rate is based on the percentage consumed). In some cases the consumption rate is measured over a time period within the first 7 hours, first 6 hours, first 5 hours, first 4 hours, first 3 hours, first 2.5 hours, first 2 hours, first 1.5 hours, first 1 hour, or first 30 mins. In some cases the consumption rate is determined in the first hour of polymerization, the second hour or polymerization, over a period of at least 15, 30, 45, or 60 minutes during the first hour, first 90 minutes, second hour, second 90 minutes, or third hour of polymerization.

The methods described herein can also include selecting to discard, withhold, reprocess through a previous manufacturing step, not process, or discontinue use of, GA or a polymeric precursor of GA (e.g., Intermediate-1), for example, if the consumption rates of two, three, or all four of the individual NCAs in the reaction mixture are not equal to, are not equivalent with, and/or are not consistent with one or more of the reference specifications (i)-(v) shown in Table 1.

Preferably, the consumption rates of all four of the individual NCAs in the reaction mixture are equal to, equivalent with, and/or are consistent with all four of the reference specifications (i)-(v) shown in Table 1.

### EXAMPLES

The invention is further described in the following examples, which do not limit the scope of invention described in the claims.

### Example 1: NCA Depletion Assay

This Example describes a method for manufacturing glatiramer acetate that includes assessing the reaction kinetics and consumption rates of the individual NCA-derivatized amino acids during Step 1 of the glatiramer acetate manufacturing process (the polymerization step) to confirm that the process will yield material suitable for commercial use as glatiramer acetate drug product. The manufacturing methods disclosed herein improve upon other methods by including steps within the manufacturing process wherein the amount of free NCA-derivatized amino acids in solution is measured and quantified, allowing the relative reactivity of each NCA to be determined over time, thereby enabling determination of reaction rates, relative kinetics, and material consumption.

Briefly, one method for assessing the amount of free NCA-amino acids in the polymerization reaction proceeds as follows. The free NCA-amino acids present in the polymerization reaction at a given time can be assessed by removing a sample, quenching the reaction with benzylamine. This converts the NCA-amino acids to their respective benzylamide derivatized amino acids. Water can then be added to precipitate the polymer (Intermediate-I) present in the quenched sample. The precipitated polymer is removed and the amount of the various benzylamide derivatized amino acids present is measured by HPLC using a set of standards. By taking samples at two time points during the polymerization reaction and measuring the amount of free NCA-amino acid present in the polymerization mixture (e.g., by converting to benzylamide derivatized amino acids) the relative rates of consumption of the amino acids can be assessed.

A reversed phase HPLC system capable of monitoring a wavelength of 215 nm, maintaining constant column temperature (40° C.±2° C.) and auto-sampler temperature (25° C.±5° C.), running a gradient method and having an in-line degasser (Waters Alliance 2695 or equivalent) is one type of system that can be used to quantify the derivatized amino acids. In the examples below, the mobile phase gradient is Mobile Phase A consisting of 0.1% Trifluoroacetic acid in purified water and Mobile Phase B consisting of 0.08% Trifluoroacetic acid in Acetonitrile, HPLC Grade.

Amino acid-benzylamide standards were used to generate standard curves for HPLC analysis. For Glu, Glu(OBn)-NHBn HCl was used as a standard and was prepared as follows. Other amino acid (Ala, Lys (OTfa) and Tyr) benzylamide derivatives can be prepared using a similar procedure.

a) Preparation of BocGlu (OBn)-NHBn: To a clear solution of Boc-Glu (OBn)-OSU (20.0 g, 46.0 mmol), in 2-MeTHF (200 mL, 10 vol) at 20-25° C., benzylamine (2 mole equivalent) was added. The reaction was allowed to stir overnight at 20-25° C. On the following day, a sample of the reaction mixture was evaporated without workup and analyzed by 1H NMR for reaction completion. Water (100 mL, 5 vol) was then added, and the resulting clear phases were separated. The organic layer was washed with 5% aqueous citric acid (100 mL, 5 vol), followed by water (100 mL, 5 vol). The organic layer was concentrated to dryness via rotary evaporation at 40-45° C., and then dried under high vacuum to afford Boc-Glu (OBn)-NH-Bn.

b) Preparation of Glu(OBn)-NHBn.HCl salt: A 500 mL three-neck RBF equipped with a magnetic stirring bar was charged with Boc-Glu(OBn)-NH-Bn (14.9 g, 38.0 mmol), followed by 1,4-dioxane (149 mL, 10 vol). The solution was charged with 4 M HCl in dioxane (47.5 mL, 5 mole. equivalent) at 20-25° C. The reaction mixture was heated to 50° C. and sampled for HPLC analysis at 0.5 hour intervals until completion. After stirring for 2 h at 50° C., the reaction mixture was cooled to room temperature. The reaction mixture was concentrated via rotary evaporation and subsequent high vacuum drying. The Glu (OBn)-NHBn hydrochloride salt was isolated with 97% AUC purity by HPLC. 1H NMR conforms to the desired structure.

Reaction of benzylamine with benzyl-protected Glu-NCA can produce both benzylamide derivatized Glu and benzylamide derivatized pyroGlu. Thus, it is useful to prepare a Pyro glutamic Acid Benzylamide standard. This can be prepared as follows. To a single-neck RBF was charged the hydrochloride salt of Glu (OBn)-NHBn (5.0 g, 14.0 mmol) and THF (50.0 mL, 10.0 vol). Triethylamine (3.0 mL, 1.5 equivalents) was added to the reaction mixture; precipitation of a white solid was observed immediately after addition. The reaction flask was placed in an oil bath preheated to 50° C. and the reaction was stirred at this temperature for 64 h. The precipitated amine salt was filtered, and the reaction mixture was concentrated to dark yellow colored oil. TLC analysis with KMnO₄ stain showed the presence of multiple spots, and the best separation was achieved with a 5% MeOH:DCM solvent mixture. Column chromatography (1-10% MeOH:DCM gradient) was performed to obtain 1.28 g (>99% AUC) of the desired compound.

Standard Preparation:

Amino acid-benzylamide standards were prepared for L-Alanine NCA (mol wt. 115.09 g/mol), L-Tyrosine NCA (mol wt. 207.18 g/mol), 5-Benzyl L-Glutamic Acid NCA (mol wt. 263.25 g/mol) and L-TFA protected Lysine NCA (mol wt. 268.19 g/mol), from the same lots of AA-NCAs as used for the Step-1 polymerization. Each individual AA-NCA was dissolved in anhydrous 1,4-dioxane to obtain an 8 mM solution. Aliquots were removed from each 8 mM solution and combined to prepare a composite standard solution containing 2 mM of each AA-NCA. Serial dilutions were prepared with anhydrous 1,4-dioxane to produce seven standards ranging in concentration from 1 mM to 0.03125 mM. In a micro-centrifuge tube, 200 μL of each AA-NCA standard solution was added to 60 μL of benzylamine followed by addition of 260 μL of purified water. The water addition was performed just prior to HPLC analysis because TFA-Lysine-benzylamide can slowly lose its TFA protecting group once water is added. Thus, it is preferable that the TFA-Lysine standard is prepared last and analyzed first to minimize the impact of this conversion. A standard curve is generated for each of the four amino acids and the linearity is evaluated by plotting the response factor versus amino acid concentration. Two reagent blanks are prepared in the same solvent ratio as used for the standard preparation. First a 1,4-dioxane and benzylamine blank is prepared followed by a water and 1,4-dioxane/benzylamine blank.

Sample Preparation:

At the start of the polymerization reaction a 50 mL reference sample (t=0) is taken prior to initiating the polymerization reaction. The density was determined for the initial reference sample (approximately 1.04 to 1.05 g/mL) which can be used for weight to volume conversions and subsequent dilution factor calculations. Upon initiation of the polymerization, samples of the reaction solution were taken periodically from a recirculation loop. Each sample aliquot, 50 mL (approximately 52 g), is quenched into 15 mL of benzylamine (density=0.981 g/mL). The benzylamine quench solution was sealed in a glass container, agitated for 30±5 minutes at room temperature and then frozen at −20° C. if analysis the analysis was not performed immediately. Upon thawing the samples, aliquots of the benzylamine-quenched reaction samples were diluted 1:1 v/v with water to precipitate the Intermediate-1 polymer. The samples were vortexed and then centrifuged at approximately 12,000 rpm for 5 minutes to separate the precipitate from the remainder of the quenched reaction solution. The supernatant volume was promptly removed and analyzed by HPLC or diluted with a solution of 50:50 1,4-dioxane:water prior to analysis, depending on the elapsed reaction time. The amounts of each of the amino acid derivative using standard curves generated using the reference samples.

Polymerization Reaction:

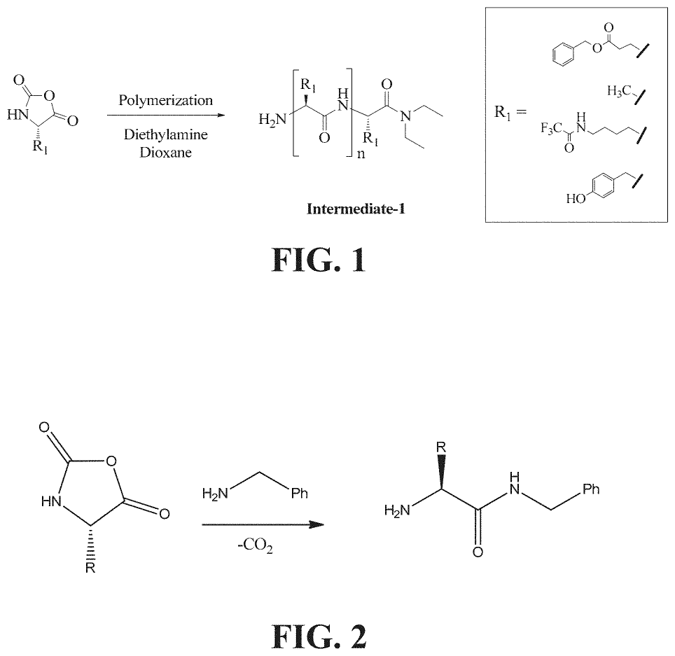

A mixture of the four amino acid-N-carboxyanhydrides (L-Alanine N-carboxy anhydride (Ala-NCA), 5-O-Benzyl-L-Glutamic acid N-carboxy anhydride (Glu-NCA), 6-N-Trifluoroacetamido-L-Lysine N-carboxy anhydride (Lys-NCA), L-Tyrosine N-carboxy anhydride (Tyr-NCA)) were polymerized using N,N-diethyl amine (DEA) initiator in dioxane solvent to produce a protected polymer referred to herein as Intermediate-1 (see FIG. 1).

Samples of the reaction mixture were collected for analysis at various time points (indicated in Table 1, below) during Step-1 (polymerization). The collected samples were contacted with benzylamine in order to derivative any unreacted NCA amino acids in the reaction mixture with benzylamine to form amino acid-benzylamide derivatives (see FIG. 2).

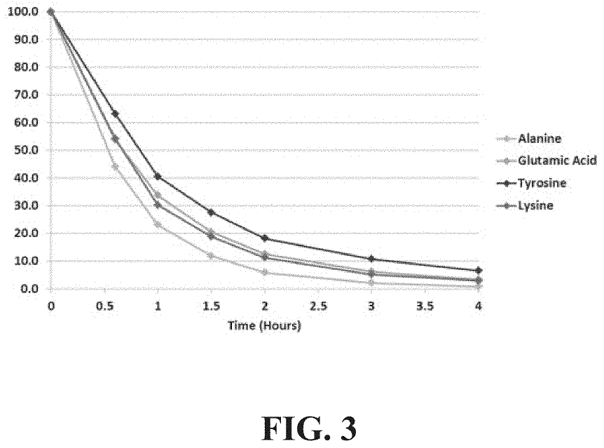

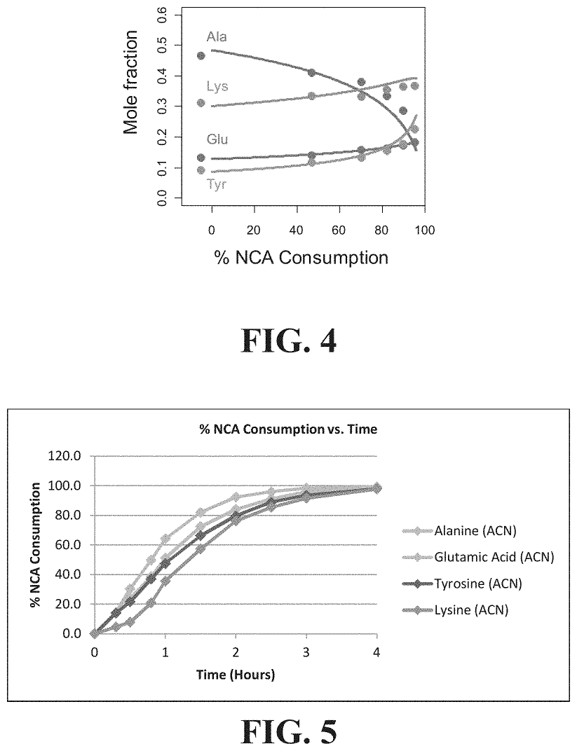

The amount of free NCAs in the solution was measured and quantified, and the percent (%) consumption of each amino acid NCA was determined. Data from a representative experiment is shown in Table 2, below, and plotted in FIG. 3 and FIG. 4. The data indicate the order in which the amino acids were consumed, with alanine being consumed faster, followed by glutamic acid and lysine, and then tyrosine. The data also show that the majority of the amino acids were consumed within the first 8 hours of the reaction.

TABLE 2
NCA Depletion Data

| | Percent (%) Consumption | | | |
|---|---|---|---|---|
| Time (hour(s)) | Ala NCA | Glu(Bn) NCA | Lys(TFA) NCA | Tyr NCA |
| 0 | 0.0 | 0.0 | 0.0 | 0.0 |
| 0.6 | 55.9 | 46.2 | 45.8 | 36.8 |
| 1 | 77.0 | 66.3 | 69.7 | 59.4 |
| 1.5 | 88.1 | 79.6 | 81.1 | 72.4 |
| 2 | 94.2 | 87.4 | 88.8 | 81.8 |
| 3 | 97.8 | 93.8 | 94.8 | 89.2 |
| 4 | 99.2 | 96.7 | 97.1 | 93.4 |
| 6 | 99.8 | 98.6 | 99.1 | 96.9 |
| 8 | 99.9 | 99.3 | 99.5 | 98.4 |
| 10 | 100.0 | 99.5 | 99.7 | 99.1 |
| 14 | 100.0 | 99.6 | 99.7 | 99.6 |
| 18 | 100.0 | 99.7 | 99.9 | 99.7 |
| 20 | 100.0 | 99.7 | 100.0 | 99.8 |
| 24 | 100.0 | 99.8 | 99.9 | 99.8 |

“Ala NCA”: Alanine-N-Carboxyanhydride;
“Glu(bn) NCA”: 5-O-Benzyl-L-Glutamic acid N-carboxy anhydride;
“Lys(TFA) NCA”: 6-N-Trifluoroacetamido-L-Lysine N-carboxy anhydride;
“Tyr NCA”: L-Tyrosine N-carboxy anhydride

Based on the reaction kinetics, it was determined that the rate of amino acid NCA consumption (depletion) relative to the other amino acid NCA in Step 1 proceeded as follows:

Percent (%) individual consumption of Ala is greater than (>) the percent individual consumption of each of Glu and Lys, and the percent of individual consumption of each of Glu and Lys was greater than (>) the percent individual consumption of Tyr, which can also be expressed as: percent (%) individual consumption of Ala>(Glu, Lys)>Tyr.

It was thus determined that a batch of Step-1 material, or the batch of glatiramer acetate produced from that Step-1 material, is suitable for processing and/or commercial release if the % individual consumption of Ala>(Glu, Lys)>Tyr.

### Example 2: Validation of Assay

This Example describes the validation of the assay using four separately generated batches of glatiramer acetate Intermediate 1, and a negative control.

Four additional Step 1 reactions were carried out and determined to meet the acceptance criteria determined in Example 1. The reactions were carried out as described in Example 1. The data are shown in Table 3, below:

TABLE 3
Additional NCA Depletion Results for Step-1 of Glatiramer
Acetate Manufacturing Process

| Acceptance | Intermediate-1 Batch Number | | | |
|---|---|---|---|---|
| Criteria | 1 | 2 | 3 | 4 |
| % Individual | Ala > | Ala > | Ala > | Ala > |
| consumption: | (Glu, Lys) > | (Glu, Lys) > | (Glu, Lys) > | (Glu, Lys) > |
| | Tyr | Tyr | Tyr | Tyr |
| % Total | ≥98% after | ≥98% after | ≥98% after | ≥98% after |
| consumption: | 24 hr | 24 hr | 24 hr | 24 hr |
| ≥98% after | | | | |
| 24 hr | | | | |

As the percent individual consumption met the acceptance criteria of Ala>(Glu, Lys)>Tyr, each of the batch numbers 1-4 were determined to be suitable for processing and/or commercial release.

A negative control was also run as follows: the Step-1 polymerization reaction was run in acetonitrile instead of dioxane, which results in different relative reactivity of the NCAs. As demonstrated in FIG. 5, the Step-1 polymerization in acetonitrile resulted in the relative kinetics of % consumption of Glu>Ala˜Tyr>Lys. The NCA reactivity in acetonitrile was clearly differentiated by the NCA depletion method, resulting in Intermediate-1 failing Intermediate-1 acceptance criteria of Ala>(Glu,Lys)>Tyr, thus deeming it not suitable to make drug substance glatiramer acetate.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-27. (canceled)

28. A method of manufacturing glatiramer acetate, the method comprising:
(a) polymerizing in a reaction mixture individual N-carboxy anhydrides (NCAs) of L-alanine (Ala), benzyl-protected L-glutamic acid (Glu), trifluoroacetic acid (TFA)-protected L-lysine (Lys), and L-tyrosine (Tyr) to generate a batch of protected copolymer;
(b) measuring the amount of two, three, or all four of the individual NCAs in the reaction mixture at two or more time points during the polymerization reaction;
(c) determining the rates of depletion of two, three, or all four of the individual NCAs in the reaction mixture using the at least two or more measurements; and
(d) processing at least a portion of the batch of the protected copolymer to generate glatiramer acetate only if one or more of the following criteria (i)-(v) is determined to have been met in step (c):
(i) the rate of depletion of NCA-Ala is greater than the rate of depletion of NCA-Glu, NCA-Lys, and/or NCA-Tyr (Ala>Glu, Lys and/or Tyr);
(ii) rate of depletion of Tyr is less than the rate of depletion of NCA-Ala, NCA-Glu, and/or NCA-Lys (Tyr<Ala, Glu, and/or Lys);
(iii) the rates of depletion of NCA-Glu and NCA-Lys are less than the rate of depletion of NCA-Ala (Ala>(Glu, Lys));
(iv) the rates of depletion of NCA-Glu and NCA-Lys are greater than the rate of depletion of NCA-Tyr ((Glu, Lys)>Tyr); and
(v) the rates of depletion of NCA-Glu and NCA-Lys are less than rate of depletion of NCA-Ala and greater than the rate of depletion of NCA-Tyr (Ala>(Glu, Lys)>Tyr).

29. The method of claim 28, wherein step (b) comprises determining the rates of depletion two of the individual NCAs in the reaction mixture.

30. The method of claim 28, wherein step (b) comprises determining the rates of depletion three of the individual NCAs in the reaction mixture.

31. The method of claim 28, wherein step (b) comprises determining the rates of depletion three of all four individual NCAs in the reaction mixture.

32. The method of claim 28, wherein step (b) comprises obtaining samples of the reaction mixture at two or more time points.

33. The method of claim 32, wherein step (b) comprises converting the NCA-amino acids in the samples to their respective benzylamide derivatized amino acids.

34. The method of claim 32, wherein step (b) comprises derivatizing unreacted NCAs in the samples.

35. The method of claim 28, wherein the step (b) comprises the use of high pressure liquid chromatography.

36. The method of claim 28, wherein the step of processing at least a portion of the batch of the protected copolymer to generate glatiramer acetate comprises treating the protected copolymer to deprotect the benzyl-protected L-glutamic acid therein and to partially depolymerize the protected copolymer.

37. The method of claim 28, further comprising determining the total depletion of NCAs in the reaction mixture after 24 hours.

38. The method of claim 28, wherein step (a) comprises polymerizing the NCAs using N,N-diethyl amine (DEA) initiator in dioxane solvent.

39. The method of claim 28, wherein step (d) comprises treating the protected copolymer to deprotect the benzyl-protected L-glutamic acid and to partially depolymerize the protected copolymer.

40. The method of claim 28, wherein step (d) comprises processing at least a portion of the batch of the protected copolymer to generate glatiramer acetate if two or more of the criteria (i)-(v) is determined to have been met in step (c), otherwise not processing the batch of protected polymer to produce glatiramer acetate.

41. The method of claim 28, wherein step (d) comprises processing at least a portion of the batch of the protected copolymer to generate glatiramer acetate if three or more of the criteria (i)-(v) is determined to have been met in step (c), otherwise not processing the batch of protected polymer to produce glatiramer acetate.

42. The method of claim 28, wherein step (d) comprises processing at least a portion of the batch of the protected copolymer to generate glatiramer acetate if four or more of the criteria (i)-(v) is determined to have been met in step (c), otherwise not processing the batch of protected polymer to produce glatiramer acetate.

43. The method of claim 28, wherein step (d) comprises processing at least a portion of the batch of the protected copolymer to generate glatiramer acetate if the rates of depletion of NCA-Glu and NCA-Lys are less than the rate of depletion of NCA-Ala and greater than the rate of depletion of NCA-Tyr (Ala>(Glu, Lys)>Tyr) in step (c).

44. The method of claim 28, wherein the step of determining the rates of depletion of two, three, or all four of the individual NCAs in the reaction mixture comprises determining the rates of depletion during the first 2 hours of polymerization reaction.

45. The method of claim 28, wherein the step of determining the rates of depletion of two, three, or all four of the individual NCAs in the reaction mixture comprises determining the rates of depletion during the first 5 hours of polymerization reaction.

46. The method of claim 28, wherein the step of determining the rates of depletion of two, three, or all four of the individual NCAs in the reaction mixture comprises determining the rates of depletion during a first time period during the polymerization reaction and during a second time period during the polymerization reaction.

47. The method of claim 32, wherein the samples are obtained during the polymerization reaction and are analyzed after the polymerization reaction is terminated.

48. The method of claim 28, wherein at least a portion of the batch of protected copolymer is deprotected and partially depolymerized

49. The method of claim 28, further comprising processing the glatiramer acetate to produce glatiramer acetate drug product by combining the glatiramer acetate with mannitol.

50. The method of claim 49, wherein the method further comprises filling a syringe with a dose of the glatiramer acetate drug product.

51. The method of claim 50, wherein the dose of the glatiramer acetate drug product is 20 mg or 40 mg.

52. The method of claim 39, wherein treating the protected copolymer to deprotect the benzyl-protected L-glutamic acid and to partially depolymerize the protected copolymer comprises treating the protected copolymer with phenol treated HBr/acetic acid.
